(19) United States
(12) Patent Application Publication
(10) Pub. No.: US 2007/0099982 A1
(43) Pub. Date: May 3, 2007

(54) Use of chp as an inhibitor of glutathione s-transferases and collagen IV

(76) Inventor: Zoser B. Salama, Berlin (DE)

Correspondence Address: JOYCE VON NATZMER;HVP LLP, 200 Madison Avenue, Suite 1901, New York, NY, 10016, US

(21) Appl. No.: 10/596,410

(22) PCT Filed: Dec. 13, 2004
(86) PCT No.: PCT/DE04/02762

§ 371 Date: Jun. 12, 2006

(30) Foreign Application Priority Data
Dec. 12, 2003 (DE) 103 59 829.4

(51) Int. Cl. A61K 31/4015

(52) U.S. Cl. 514/423

(57) ABSTRACT

The invention relates to the use of cis-hydroxy proline for the inhibition of glutathione s-transferases and/or collagen IV, in addition to a method which is used to lower the concentration of glutathione s-transferase and/or collagen IV in vitro or in vivo, in addition to anti-collagen IV/collagen IV-lowerers or glutathione s-transferase agents/glutathione s-transferase-lowerers.

The invention relates to the use of cis-hydroxyproline (CHP) to inhibit glutathione S transferases and/or collagen IV, to a method of lowering the concentration or reducing the activity of glutathione S transferases and/or collagen IV in vitro or in vivo, and to anti-collagen IV agents/collagen IV-lowering agents or glutathione S transferase agents/glutathione S transferase-lowering agents.

A number of options of treating metabolic diseases, autoimmune diseases, neurological diseases and/or tumors have been described in the prior art. Frequently, the above diseases appear in combination, but there are no agents available that could treat the above diseases in combination.

More specifically, this is because no multifunctional targets associated with the development of metabolic diseases, autoimmune diseases, neurological diseases, as well as tumor diseases and/or other pathological changes, have been detected. Accordingly, there are no methods or agents available that would act on such targets in such a way that development of the above-mentioned diseases would be prevented in a combined fashion.

In spite of the inconsistency in the prior art with respect to key targets associated with a plurality of diseases, some biomolecules present in organisms have been described, for which a relationship to pathological changes in an organism is being discussed in the literature, such as neutral endopeptidase (NEP) and other metallo-endopeptidases.

More specifically, so-called marker molecules are concerned, whose presence within a specific concentration range can provide evidence as to specific changes in an organism that are associated with a disease.

The object of the invention was to detect new key targets and provide pharmaceutical agents and methods by means of which the activity or, respectively, the concentration of key targets could be inhibited or suppressed, i.e., provide agents which could be used as key target-lowering agents.

Surprisingly, it was found that cis-hydroxyproline can be used to inhibit the concentration or activity of the collagen IV and/or glutathione S transferase key targets. More specifically, cis-hydroxyprolines (CHPs) in the meaning of the invention are cis-hydroxy-L-proline and salts thereof.

CHP can be used as isolated compound or as a mixture with other compounds or as a prodrug which is converted into the free CHP form in the body of an organism. Inhibition or suppression of GST, especially αGST, and collagen IV can be effected in vitro and in vivo. For example, in vivo inhibition can be inhibition in an organism, e.g. in an animal or in a human individual, and in vitro inhibition can be, for example, inhibition in a tissue structure, e.g. a liver structure in a cell-biological culture vessel.

Obviously, inhibition can also be applied in extracorporeal circulations, e.g. in an artificial liver connected to an animal or human patient.

CHP can have an inhibiting effect both in vitro and in vivo. In an in vivo system, e.g. a patient, oral or intravenous or intramuscular application of CHP can be envisaged. In in vitro systems, direct supply of CHP in the form of a powder or solution or in combination with carriers, such as liposomes, into the in vitro system, or previous mixing with a culture solution, e.g. a nutrient solution, and subsequent incorporation in the system can be envisaged, for example.

GST inhibition or lowering and/or collagen IV inhibition or lowering in a cell culture or in an organism has a number of consequences. In organisms or in vitro cultures, for example, GST is capable of binding GSH so as to prepare the latter for extracellular transport. In the event of a tumor cell, this would imply the following: GST binds oncogens or other components of the tumor cell to GSH, conveying them into the extracellular region, which—among other things—gives rise to the spreading effect and, as a consequence, formation of metastases. As a result of increased GSH binding, the latter is no longer available for other cellular processes, and this gives rise to pathological changes in the cell. In addition, binding of tumor cell fragments results in a different way of information processing within the cell, so that functions proceed in a different way, thereby initiating or promoting transformation of the cell. Moreover, the processes mentioned above promote apoptosis.

However, higher tolerance to carcinogens and inhibition of carcinogenesis are not the only consequences of inhibition effected by CHP. Other secondary responses of such inhibition comprise e.g. therapy or alleviation of autoimmune diseases, regeneration of cells following chemotherapy or in parallel with chemotherapy, relieve of the ageing process by removing interfering radicals, treatment of infectious diseases as well as metabolic diseases, especially of the liver, pancreas, intestine and/or stomach.

In a preferred fashion, such secondary processes of GST inhibition are associated with other chemical secondary processes of collagen IV inhibition. In particular, the secondary processes of collagen IV inhibition result from the fact that tumor cells dock via the main collagen domain of this glycolprotein, thus infiltrating and penetrating the cells. However, collagen inhibition not only results in diminished metastasizing and infiltration and invasion in tumor diseases, but also exhibits therapeutic effects in all inflammatory diseases wherein normal tissue is reconstructed into connective tissue, e.g. in lung fibrosis, liver cirrhosis, pancreatic fibrosis and/or glomerulosclerosis. Furthermore, collagen IV inhibition shows a positive influence on scleroderma/Marfan syndrome, vascular diseases, metabolic diseases, autoimmune diseases, and neurological diseases wherein nervous tissue is turned into connective tissue, so-called glioses, as is the case in Alzheimer's disease, for example. In addition to inhibiting collagen IV by CHP, it is obviously possible—particularly in the last-mentioned diseases—to administer parallel medications inducing fibrosis, e.g. bleomycin/busulfan, in the form of a supportive/additive therapy.

The invention also relates to a method of inhibiting collagen IV and/or GST in an organism and/or in a sample, in which method the organism or a sample is contacted with CHP. For example, the method can be used in a combination therapy, by means of which cells in an organism regenerate following chemotherapy. For example, contacting of CHP with the organism or the sample to be treated can be effected orally, subcutaneously, intravenously, intramuscularly, intraperitoneally, vaginally, rectally, topically and/or sublingually.

The invention also relates to an anti-collagen IV agent and/or anti-GST agent or collagen IV- or GST-lowering agent comprising CHP, optionally together with standard auxiliary agents. More specifically, these standard auxiliary agents are pharmaceutically acceptable carriers, adjuvants and/or vehicles, said carriers being selected from the group comprising fillers, diluents, binders, humectants, disintegrants, dissolution retarders, absorption enhancers, wetting agents, adsorbents and/or lubricants. The collagen IV-lowering agent or inhibitor or the GST-lowering agent or inhibitor comprising CHP can be prepared and/or used in the form of a gel, poudrage, powder, tablet, sustained-release tablet, premix, emulsion, brew-up formulation, drops, concentrate, infusion solutions, granulate, syrup, pellet, bolus, capsule, aerosol, spray and/or inhalant. In a preferred fashion, CHP is present in a formulation at a concentration of from 0.1 to 99.5, preferably from 0.5 to 95, and more preferably from 1 to 80 wt.-%. In a particularly preferred fashion the formulation is an infusion solution wherein CHP is present in a range of from 1 to 2 wt.-%.

In another embodiment of the invention, CHP is employed in overall amounts of from 0.05 to 1000 mg per kg body weight, preferably from 5 to 450 mg per kg body weight per 24 hours.

The collagen IV inhibitor or GST inhibitor or CHP alone can be used in such a way that 0.1 to 100 g is administered per day and patient. Of course, splitting the daily dose and contacting the correspondingly split amount 2, 4, 6 or 10 times or more with the organism can also be envisaged.

Inhibition of collagen IV and/or GST, preferably αGST, by CHP is preferably used in the treatment of (i) inflammations, especially preferably (ii) autoimmune diseases.

(i) Inflammations in the meaning of the invention are reactions of the organism, mediated by the connective tissue and blood vessels, to an external or internally triggered inflammatory stimulus, with the purpose of eliminating or inactivating the latter and repairing the tissue lesion caused by said stimulus. A triggering effect is caused by mechanical stimuli (foreign bodies, pressure, injury) and other physical factors (ionizing radiation, UV light, heat, cold), chemical substances (alkaline solutions, acids, heavy metals, bacterial toxins, allergens, and immune complexes), and pathogens (microorganisms, worms, insects), or pathologic metabolites, derailed enzymes, malignant tumors. The process begins with a brief arteriolar constriction (as a result of adrenaline effect), with inadequate circulation and tissue alteration, followed by development of classical local inflammatory signs (cardinal symptoms, according to GALEN and CELSUS), i.e., from reddening (=rubor; vascular dilation caused by histamine), heat (=calor; as a result of local increase of metabolism), swelling (=tumor; as a result of secretion of protein-rich liquor from vessel walls changed by histamine, among other things, supported by decelerated blood circulation in the sense of a prestasis up to stasis), pain (=dolor; as a result of increased tissue tension and algogenic inflammation products, e.g. bradykinin), and functional disorders (=functio laesa). The process is accompanied by disorders in the electrolyte metabolism (transmineralization), invasion of neutrophilic granulocytes and monocytes through the vessel walls (cf., leukotaxis), with the purpose of eliminating the inflammatory stimulus and the damaged to necrotic cells (phagocytosis); furthermore, invasion of lymphocyte effector cells, giving rise to formation of specific antibodies against the inflammatory stimulus (immune reaction), and of eosinophiles (during the phase of healing or—at a very early stage—in allergic-hyperergic processes). As a result of the activation of the complement system occurring during the reaction, fragments (C3a and C5a) of this system are liberated which—like histamine and bradykinin—act as inflammation mediators, namely, in the sense of stimulating the chemotaxis of the above-mentioned blood cells; furthermore, the blood coagulation is activated. As a consequence, damage (dystrophia and coagulation necrosis) of the associated organ parenchyma occurs. Depending on the intensity and type of the inflammation, the overall organism responds with fever, stress (cf., adaptation syndrome), leukocytosis and changes in the composition of the plasma proteins (acute-phase reaction), giving rise to an accelerated erythrocyte sedimentation. Preferred inflammations in the meaning of the invention are suppurative, exudative, fibrinous, gangrenescent, granulomatous, hemorrhagic, catarrhal, necrotizing, proliferative or productive, pseudomembranous, serous, specific and/or ulcerous inflammations.

(ii) Autoimmune diseases in the meaning of the invention are diseases entirely or partially due to the formation of autoantibodies and their damaging effect on the overall organism or organ systems, i.e., due to autoaggression. A classification into organ-specific, intermediary and/or systemic autoimmune diseases can be made. Preferred organ-specific autoimmune disease are HASHIMOTO thyroiditis, primary myxedema, thyrotoxicosis (BASEDOW disease), pernicious anemia, ADDISON disease, myasthenia gravis and/or juvenile diabetes mellitus. Preferred intermediary autoimmune diseases are GOODPASTURE syndrome, autoimmune hemolytic anemia, autoimmune leukopenia, idiopathic thrombocytopenia, pemphigus vulgaris, sympathetic ophthalmia, primary bile cirrhosis, autoimmune hepatitis, ulcerative colitis and/or SJÖGREN syndrome. Preferred systemic autoimmune diseases are rheumatoid arthritis, rheumatic fever, systemic lupus erythematosus, dermatomyositis/polymyositis, progressive systemic sclerosis, WEGENER granulomatosis, panarteritis nodosa and/or hypersensitivity angiitis. Typical autoimmune diseases are thyrotoxicosis, thyroid-caused myxedema, HASHIMOTO thyroiditis, generalized endocrinopathy, pernicious anemia, chronic gastritis type A, diseases of single or all corpuscular elements of the blood (for example, autoimmune hemolytic anemia, idiopathic thrombocytopenia or thrombocytopathy; idiopathic leukopenia or agranulocytosis), pemphigus vulgaris and pemphigoid, sympathetic ophthalmia, and numerous forms of uveitis, primarily biliary liver cirrhosis and chronic aggressive autoimmune hepatitis, diabetes mellitus type I, CROHN disease and ulcerative colitis, SJÖGREN syndrome, ADDISON disease, lupus erythematosus disseminatus and discoid form of said disease, as dermatomyositis and scleroderma, rheumatoid arthritis (=primarily chronic polyarthritis), antiglomerular basement membrane nephritis. The basis is an aggressive immune reaction due to breakdown of the immune tolerance to self-determinants and a reduction of the activity of T suppressor cells (with lymphocyte marker T8) or an excess of T helper cells (with lymphocyte marker T4) over the suppressor cells; furthermore, formation of autoantigens is possible e.g. by coupling of host proteins to haptens (e.g. drugs), by ontogenetic tissue not developing until self-tolerance has developed, by protein components demasked as a result of conformational changes of proteins in connection with e.g. infection by viruses or bacteria; and by new proteins formed in association with neoplasias.

In a preferred embodiment the cancerous disease or tumor being treated or prophylactically prevented, or whose reappearance is prevented, is selected from the group of cancerous diseases or tumor diseases of the ear-nose-throat region, of the lungs, mediastinum, gastrointestinal tract, urogenital system, gynecological system, breast, endocrine system, skin, bone and soft-tissue sarcomas, mesotheliomas, melanomas, neoplasms of the central nervous system, cancerous diseases or tumor diseases during infancy, lymphomas, leukemias, paraneoplastic syndromes, metastases with unknown primary tumor (CUP syndrome), peritoneal carcinomatoses, immunosuppression-related malignancies and/or tumor metastases.

More specifically, the tumors may comprise the following types of cancer: adenocarcinoma of breast, prostate and colon; all forms of lung cancer starting in the bronchial tube; bone marrow cancer, melanoma, hepatoma, neuroblastoma; papilloma; apudoma, choristoma, branchioma; malignant carcinoid syndrome; carcinoid heart disease, carcinoma (for example, Walker carcinoma, basal cell carcinoma, squamobasal carcinoma, Brown-Pearce carcinoma, ductal carcinoma, Ehrlich tumor, in situ carcinoma, cancer-2 carcinoma, Merkel cell carcinoma, mucous cancer, non-parvicellular bronchial carcinoma, oat-cell carcinoma, papillary carcinoma, scirrhus carcinoma, bronchioalveolar carcinoma, bronchial carcinoma, squamous cell carcinoma and transitional cell carcinoma); histiocytic functional disorder; leukemia (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia); malignant histiocytosis, Hodgkin disease, non-Hodgkin lymphoma, solitary plasma cell tumor; reticuloendotheliosis, chondroblastoma; chondroma, chondrosarcoma; fibroma; fibrosarcoma; giant cell tumors; histiocytoma; lipoma; liposarcoma; leukosarcoma; mesothelioma; myxoma; myxosarcoma; osteoma; osteosarcoma; Ewing sarcoma; synovioma; adenofibroma; adenolymphoma; carcinosarcoma, chordoma, craniopharyngioma, dysgerminoma, hamartoma; mesenchymoma; mesonephroma, myosarcoma, ameloblastoma, cementoma; odontoma; teratoma; thymoma, chorioblastoma; adenocarcinoma, adenoma; cholangioma; cholesteatoma; cylindroma; cystadenocarcinoma, cystadenoma; granulosa cell tumor; gynadroblastoma; hidradenoma; islet-cell tumor; Leydig cell tumor; papilloma; Sertoli cell tumor, theca cell tumor, leiomyoma; leiomyosarcoma; myoblastoma; myoma; myosarcoma; rhabdomyoma; rhabdomyosarcoma; ependymoma; ganglioneuroma, glioma; medulloblastoma, meningioma; neurilemmoma; neuroblastoma; neuroepithelioma, neurofibroma, neuroma, paraganglioma, non-chromaffin paraganglioma, angiokeratoma, angiolymphoid hyperplasia with eosinophilia; sclerotizing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phylloides; hemangiosarcoma; lymphangiosarcoma; myxosarcoma, ovarian carcinoma; sarcoma (for example, Ewing sarcoma, experimentally, Kaposi sarcoma and mast cell sarcoma); neoplasms (for example, bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of the head and neck, of the central nervous system, neoplasms of the hearing organ, pelvis, respiratory tract and urogenital tract); neurofibromatosis and cervical squamous cell dysplasia.

In another preferred embodiment the cancerous disease or tumor being treated or prophylactically prevented, or whose reappearance is prevented, is selected from the group of cancerous diseases or tumor diseases comprising cells including the MUC1 in the definition according to the invention, selected from the group of: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs, comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancies such as Kaposi sarcoma, AIDS-associated lymphomas, AIDS-associated lymphomas of the central nervous system, AIDS-associated Hodgkin disease, and AIDS-associated anogenital tumors, transplantation-related malignancy, metastasized tumors comprising brain metastases, lung metastases, liver metastases, bone metastases, pleural and pericardial metastases, and malignant ascites.

In another preferred embodiment the cancerous disease or tumor being treated or prophylactically prevented, or whose reappearance is prevented, is selected from the group comprising cancerous diseases or tumor diseases such as mammary carcinomas, gastrointestinal tumors, including colon carcinomas, stomach carcinomas, large intestine cancer and small intestine cancer, pancreas carcinomas, ovarian carcinomas, liver carcinomas, lung cancer, renal cell carcinomas, multiple myelomas.

Without intending to be limiting, the invention will be explained in more detail with reference to the following example.

### EXAMPLE

Inhibition of Collagen IV by CHP in Humans

Table 1 shows the results of the determination of collagen IV from various healthy subjects as a function of time (days). CHP was repeatedly administered over 14 days, using 4×2 g of CHP per day.

TABLE 1
Concentration of collagen IV in serum samples from healthy subjects
Collagen IV

| | Time (days) | | | | | |
|---|---|---|---|---|---|---|
| Individual | 0 | 7 | 13 | 13.25 | 14 | 17 |
| 01 | 100.1 | 76.66 | 67.03 | 67.62 | 68.2 | 72.21 |
| 02 | 112.4 | 73.88 | 84.83 | 73.32 | 83.23 | 76.66 |
| 03 | 125.5 | 94.89 | 119.4 | 100.1 | 99.05 | 105.7 |
| 04 | 129 | 106.7 | 114.9 | 110.8 | 122 | 134.1 |
| 05 | 136.1 | 80.54 | 91.24 | 86.44 | 91.76 | 101.6 |
| 06 | 113.9 | 102.1 | 103.2 | 99.57 | 113.9 | 93.85 |
| 07 | 103.7 | 88.58 | 84.3 | 79.43 | 83.76 | 62.95 |
| 08 | 106.2 | 98.01 | 101.1 | 93.85 | 100.6 | 85.9 |
| 09 | 126.5 | 92.8 | 95.93 | 85.37 | 90.18 | 89.11 |
| 10 | 134.6 | 134.1 | 144.8 | 141.2 | 148.3 | 137.1 |
| 11 | 112.4 | 85.37 | 102.7 | 99.57 | 91.76 | 69.37 |
| 12 | 84.3 | 82.69 | 88.04 | 77.21 | 80.54 | 92.8 |
| N | 12 | 12 | 12 | 12 | 12 | 12 |
| MEAN | 115.39 | 93.03 | 99.79 | 92.87 | 97.77 | 93.45 |
| SDEV | 15.52 | 16.40 | 20.05 | 19.87 | 21.57 | 23.54 |

FIG. 1 shows the inhibition of collagen IV in the course of several days following administration of CHP (4×2.0 g CHP/day; 14 days). An individual distribution of the serum concentrations is shown in FIG. 2.

Inhibition of α-Glutathione S Transferase

The results of the GST determination are shown in Table 2.

TABLE 2
Concentration of α-glutathione S transferase
in serum samples from healthy subjects

| | | Glutathione S | | | | |
|---|---|---|---|---|---|---|
| | | transferase | | | | |
| | Time (days) | | | | | |
| Individual | 0 | 7 | 13 | 13.25 | 14 | 17 |
| 01 | 0.465 | 0.1328 | 0.279 | 0.1195 | 0.1062 | 0.093 |
| 02 | 0.5581 | 0.2657 | 0.4916 | 0.3055 | 0.1594 | 0.2125 |
| 03 | 0.5581 | 0.3985 | 0.2657 | 0.2258 | 0.1195 | 0.186 |
| 04 | 0.2923 | 0.2258 | 0.1594 | 0.1461 | 0.05314 | 0.1461 |
| 05 | 0.1461 | 0.186 | 0.2524 | 0.1993 | 0.2657 | 0.1195 |
| 06 | 1.117 | 0.2258 | 0.2524 | 0.2125 | 0.2657 | 0.2391 |
| 07 | 0.4783 | 0.2524 | 0.2923 | 0.2657 | 0.3454 | 0.186 |
| 08 | 0.1993 | 0.3587 | 0.2258 | 0.2125 | 0.279 | 0.1062 |
| 09 | 0.8107 | 1.223 | 0.2391 | 0.1195 | 0.2258 | 0.3188 |
| 10 | 0.3055 | 0.279 | 0.2258 | 0.2391 | 0.2524 | 0.1993 |
| 11 | 0.1993 | 0.3321 | 0.1727 | 0.1727 | 0.1594 | 0.093 |
| 12 | 0.3985 | 0.5847 | 0.2258 | 0.186 | 0.2391 | 0.2391 |
| N | 12 | 12 | 12 | 12 | 12 | 12 |
| MEAN | 0.46 | 0.37 | 0.26 | 0.20 | 0.21 | 0.18 |
| SDEV | 0.28 | 0.29 | 0.08 | 0.06 | 0.09 | 0.07 |

The GST values after administration of CHP at a dose of 4×2.0 g CHP/day over 14 days are shown in FIG. 3. Furthermore, the individual distribution of GST following administration of CHP in several subjects is illustrated in FIG. 4.

1.-12. (canceled)

13. A method for inhibiting collagen IV and/or glutathione S transferase (GST) comprising
administering to cells or a patient benefiting from such inhibition cis-hydroxyproline (CHP) in a collagen IV and/or glutathione S transferase (GST) inhibiting effective amount.

14. The method of claim 13, wherein said inhibition is effected in vitro or in vivo.

15. The method of claim 13, wherein CHP is in form of a gel, poudrage, powder, tablet, sustained-release tablet, premix, emulsion, brew-up formulation, infusion solution, drops, concentrate, granulate, syrup, pellet, bolus, capsule, aerosol, spray and/or inhalant.

16. The method of claims 13, wherein a formulation comprising CHP at a concentration of from 0.1 to 99.5 wt. % is administered.

17. The method of claims 16, wherein a formulation comprising CHP at a concentration of from 0.5 to 95 wt. % is administered.

18. The method of claims 17, wherein a formulation comprising CHP at a concentration of from 1 to 80 wt. % is administered.

19. The method of claim 15, wherein CHP is in form of a infusion solutions having a concentration of 1 to 2 wt. % of CHP.

20. The method of claims 13, wherein CHP is administered at amounts of from 0.05 to 1000 mg per kg body weight per 24 hours.

21. The method of claims 20, wherein CHP is administered at amounts of from 5 to 450 mg per kg body weight per 24 hours.

22. A method for the inhibition of glutathione S transferase and/or collagen IV in an in vivo or in vitro system, wherein the system is contacted with CHP.

23. The method of claim 22, wherein the system is an in vivo systems and contacting is effected orally, vaginally, rectally, nasally, subcutaneously, intravenously, intramuscularly, regionally, intraperitoneally and/or topically.

24. An anti-collagen IV and/or anti-GST agent comprising CHP, optionally together with a pharmaceutically tolerable carrier, wherein said agent inhibits collagen IV and/or GST.

25. The agent of claim 24, wherein the carrier is a filler, diluent, binder, humectant, disintegrant, dissolution retarder, absorption enhancer, wetting agent, adsorbent and/or lubricant.

26. The agent of claim 24, wherein said carrier is a liposome, siosome and/or niosome.
